(19) United States
(12) Patent Application Publication
(10) Pub. No.: US 2023/0241314 A1
(43) Pub. Date: Aug. 3, 2023

(54) EVENT-DRIVEN COMPENSATED INSULIN DELIVERY OVER TIME

(71) Applicant: CAPILLARY BIOMEDICAL, INC., Irvine, CA (US)

(72) Inventors: Mark C. Estes, Malibu, CA (US); David S. Gillett, San Diego, CA (US); Miami Shafeeq, Irvine, CA (US)

(21) Appl. No.: 18/011,060

(22) PCT Filed: Jun. 18, 2021
(86) PCT No.: PCT/US21/38028

§ 371 Date: Dec. 16, 2022

Related U.S. Application Data
(60) Provisional application No. 63/041,005, filed on Jun. 18, 2020.

(51) Int. Cl. A61M 5/172; A61M 5/142; G05B 13/02; G05B 13/04

(52) U.S. Cl. CPC A61M 5/1723; A61M 5/14248; G05B 13/0285; G05B 13/048; A61M 2202/0486

(57) ABSTRACT

Embodiments of systems and methods for delivering a medicament using a pump are provided. The methods comprise inserting a first cannula into subcutaneous tissue. Medicament is delivered from a medicament pump through the first cannula according to a dosing protocol. The first cannula can removed after a period of time and a second cannula can be inserted. The method comprises modifying the dosing protocol based upon a cannula change indicator, the modifying comprising performing neural network calculations utilizing previously calculated error data, resulting in a modified dosing protocol. Medicament is delivered from the medicament pump through the second cannula according to the modified dosing protocol.

### CROSS REFERENCE TO RELATED APPLICATIONS

This application claims the benefit of U.S. Provisional Patent Application No. 63/041,005, filed Jun. 18, 2020, the entire disclosure of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

Generally, described herein are devices for delivering therapeutic fluids and more particularly to portable infusion devices and methods that can be used to subcutaneously or percutaneously deliver these fluids safely and simply to a mammalian patient. Even more particularly, described herein is a subcutaneous insulin delivery based on AID (automated insulin delivery) enabled by dosing algorithms controlled by glycemic data derived from CGM (continuous glucose monitoring) sensors.

### BACKGROUND

During insulin delivery of insulin based on AID (automated insulin delivery) algorithms, the amount of insulin provided at the insertion site may rise over time to compensate for decreased insulin perfusion/absorption rates (decreased “insulin sensitivity”) at the inserted infusion site. The increase in insulin provided at the insertion site may be particularly common with the use of extended-wear in-dwelling catheters (infusion sets or ports) when the infusion set is worn for extended periods, e.g., for greater than 3 days, such as greater than 5 days, such as 5-10 days.

Current dosing algorithms, however, are driven by recent dosage history. As a result, if a first extended-wear in-dwelling catheter is removed and a second replacement catheter is inserted in a new site, the dosing algorithm may deliver too much insulin (i.e., the algorithm may deliver the higher amount of insulin that was delivered just prior to removal of the first catheter). Because the new site will not yet have become desensitized to insulin, the potential exists to over-deliver insulin, which may cause a hypoglycemic excursion.

A system to compensate for this potential hypoglycemic excursion is therefore desired.

### SUMMARY OF THE DISCLOSURE

In a first aspect, in some embodiments, a method of providing a medicament is provided. The method comprises inserting a first cannula into subcutaneous tissue; delivering the medicament from a medicament pump through the first cannula according to a dosing protocol; removing the first cannula from the subcutaneous tissue after a period of time; inserting a second cannula into subcutaneous tissue; modifying the dosing protocol based upon a cannula change indicator, the modifying comprising performing neural network calculations utilizing previously calculated error data, resulting in a modified dosing protocol; and delivering medicament from the medicament pump through the second cannula according to the modified dosing protocol.

In some embodiments, the medicament is insulin.

The period of time can be greater than 1 day. In some embodiments, the period of time is greater than 3 days. The period of time can be 5-10 days.

In some embodiments, modifying the dosing protocol comprises lowering an initial amount of insulin delivered relative to an amount of insulin delivered just prior to removing the first cannula.

The cannula change indicator can be a pump rewind. The cannula change indicator can be an insulin reservoir or cartridge change. The cannula change indicator can be a cannula or tubing prime. The cannula change indicator can be a user input. The cannula change indicator can be derived from applying and activating a new patch pump.

Modifying the dosing protocol can include modifying a historical setting according to previous cannula changes. Modifying the dosing protocol can include decreasing a basal rate. Modifying the dosing protocol can include decreasing a bolus dose infusion. Modifying the dosing protocol can include decreasing an insulin sensitivity setting. Modifying the dosing protocol can include modifying an algorithm gain or algorithm dampening. Modifying the dosing protocol can include adjusting an insulin action time. Modifying the dosing protocol can include adjusting a fuzzy logic control gain based on a duration of time since the change set indicator. Modifying the dosing protocol can include modifying a carbohydrate ratio. Modifying the dosing protocol can include modifying a blood glucose target.

In some embodiments, the dosing protocol comprises retaining past performance history data of compensation-derived delivery error conditions and/or states. The modified dosing protocol can be based on algorithmic computation including the retained error conditions and/or states.

The dosing protocol can comprise retaining historical performance data and modifying the dosing protocol comprises utilizing the historical performance data.

In some embodiments, the modified dosing protocol comprises using only historical data relevant to one or more previous cannula changes. The modified dosing protocol can comprise using only historical data from a time period surrounding prior cannula change events.

In some embodiments, the dosing protocol comprises using a PID based calculation to determine a dosage of medicament to be delivered.

The dosing protocol can comprise using an extended Kalman filter (EKF).

The modified dosing protocol can comprise addition of a new matrix upon recognition of a cannula change indicator.

The dosing protocol can comprise dynamically defining a low glucose threshold.

In some embodiments, the dosing protocol comprises dynamically defining a pump control prohibition for control of a delivery suspension time period.

The dosing protocol can comprise dynamically controlling a look-ahead predictive period.

In some embodiments, the dosing protocol comprises dynamically controlling default high and low glucose limit settings.

The dosing protocol can comprise dynamically changing a threshold sensitivity control of minimum delivery rate and maximum delivery rate.

In some embodiments, the dosing protocol comprises dynamically changing a threshold sensitivity control of at least one of CGM derived measurement and finger stick glucose measurement.

The dosing protocol can comprise dynamically defining an insulin response array.

In some embodiments, the dosing protocol comprises overriding user notification settings upon occurrence of a predicted or real-time control limit threshold violation.

In another aspect, in some embodiments, a method of delivering a medicament is provided. The method comprises inserting a first cannula into subcutaneous tissue; delivering the medicament from a medicament pump through the first cannula according to a dosing protocol, wherein the protocol comprises retaining past performance history data; removing the first cannula from the subcutaneous tissue after a period of time; inserting a second cannula into subcutaneous tissue; modifying the dosing protocol based upon a cannula change indicator and the past performance history data, resulting in a modified dosing protocol; and delivering medicament from the medicament pump through the second cannula according to the modified dosing protocol.

In some embodiments, the medicament is insulin.

The period of time can be greater than 1 day. The period of time can be greater than 3 days. The period of time can be 5-10 days.

In some embodiments, modifying the dosing protocol comprises lowering an initial amount of insulin delivered relative to an amount of insulin delivered just prior to removing the first cannula.

The cannula change indicator can be a pump rewind. The cannula change indicator can be an insulin reservoir or cartridge change. The cannula change indicator can be a cannula or tubing prime. The cannula change indicator can be a user input. The cannula change indicator can be derived from applying and activating a new patch pump.

Modifying the dosing protocol can include modifying a historical setting according to previous cannula changes. Modifying the dosing protocol can include decreasing a basal rate. Modifying the dosing protocol can include decreasing a bolus dose infusion. Modifying the dosing protocol can include decreasing an insulin sensitivity setting. Modifying the dosing protocol can include modifying an algorithm gain or algorithm dampening. Modifying the dosing protocol can include adjusting an insulin action time. Modifying the dosing protocol can include adjusting a fuzzy logic control gain based on a duration of time since the change set indicator. Modifying the dosing protocol can include modifying a carbohydrate ratio. Modifying the dosing protocol can include modifying a blood glucose target.

In some embodiments, the dosing protocol comprises retaining past performance history data of compensation-derived delivery error conditions and/or states. The modified dosing protocol can be based on algorithmic computation including the retained error conditions and/or states.

The modified dosing protocol can comprise using only relevant historical data. In some embodiments, the modified dosing protocol comprises using only historical data related to prior cannula change events.

The modified dosing protocol can comprise using neural network calculations utilizing previous calculated error data to determine dosage of medicament to be delivered.

In some embodiments, the dosing protocol comprises using a PID based calculation to determine a dosage of medicament to be delivered.

The dosing protocol can comprise using an extended Kalman filter (EKF).

The modified dosing protocol can comprise addition of a new matrix upon recognition of a cannula change indicator.

The dosing protocol can comprise dynamically defining a low glucose threshold.

In some embodiments, the dosing protocol comprises dynamically defining a pump control prohibition for control of a delivery suspension time period.

The dosing protocol can comprise dynamically controlling a look-ahead predictive period.

In some embodiments, the dosing protocol comprises dynamically controlling default high and low glucose limit settings.

The dosing protocol can comprise dynamically changing a threshold sensitivity control of minimum delivery rate and maximum delivery rate.

In some embodiments, the dosing protocol comprises dynamically changing a threshold sensitivity control of at least one of CGM derived measurement and finger stick glucose measurement.

The dosing protocol can comprise dynamically defining an insulin response array.

In some embodiments, the dosing protocol comprises overriding user notification settings upon occurrence of a predicted or real-time control limit threshold violation.

In yet another aspect, a subcutaneous medicament delivery system is provided. The system comprises a pump; a cannula attached to the pump; and a controller, the controller configured to: automatically calculate a medicament dosage protocol based upon a blood glucose level; modify the medicament dosage protocol when an event driven signal indicative of an infusion cannula change is detected; and use neural network calculations utilizing calculated error data to modify the medicament dosage protocol.

The medicament can be insulin. The medicament can be glucagon.

In some embodiments, modifying the medicament dosage comprises temporarily reducing a calculated dose of insulin.

Modifying the medicament dosage can comprise temporarily increasing a calculated dose of glucagon.

The step of automatically calculating can be performed with a Kalman filter.

In some embodiments, the step of automatically calculating is performed with a model predictive control (MPC).

The controller can be a PID feedback controller.

The controller can be a fuzzy logic controller.

In some embodiments, modifying the medicament dosing protocol comprises modifying a historical setting according to previous cannula changes.

Modifying the medicament dosing protocol can comprise decreasing a basal rate.

In some embodiments, modifying the medicament dosing protocol comprises modifying a bolus infusion.

Modifying the medicament dosing protocol can comprise modifying a carbohydrate ratio.

In some embodiments, modifying the medicament dosing protocol comprises modifying a glucose target.

The controller can comprise a PID feedback-driven controller, a Kalman equation-driven controller, an extended Kalman filter controller, a regression tree-driven controller, a recurrent neural network-driven controller, a feed-forward neural network-driven controller, a support vector machine-driven controller, a self-organizing map-driven controller, a Gaussian process-driven controller, a genetic algorithm and program-driven controller, or a deep neural network-driven controller.

The controller can be configured to retain historical performance data and use retained historical performance data to modify the medicament dosage protocol.

In some embodiments, the controller is configured to retain historical performance data relevant to one or more previous cannula changes, and only use the relevant historical performance data to modify the medicament dosage protocol.

The relevant historical performance data can comprise data from a set time period surrounding one or more previous cannula changes.

In another aspect, in some embodiments, a subcutaneous medicament delivery system is provided. The system comprises a pump; a cannula attached to the pump; and a controller, the controller configured to: automatically calculate a medicament dosage protocol based upon a blood glucose level; modify the medicament dosage protocol when an event driven signal indicative of an infusion cannula change is detected; and retain historical performance data and use retained historical performance data to modify the medicament dosage protocol.

In some embodiments, the controller is configured to use relevant historical performance data relevant to one or more previous infusion cannula changes.

Relevant historical performance data can comprise data from a time period surrounding one or more previous infusion cannula changes.

The medicament can be insulin. The medicament can be glucagon.

In some embodiments, modifying the medicament dosage comprises temporarily reducing a calculated dose of insulin.

Modifying the medicament dosage can comprise temporarily increasing a calculated dose of glucagon.

The step of automatically calculating can be performed with a Kalman filter.

In some embodiments, the step of automatically calculating is performed with a model predictive control (MPC).

The controller can be a PID feedback controller.

The controller can be a fuzzy logic controller.

In some embodiments, modifying the medicament dosing protocol comprises modifying a historical setting according to previous cannula changes.

Modifying the medicament dosing protocol can comprise decreasing a basal rate.

In some embodiments, modifying the medicament dosing protocol comprises modifying a bolus infusion.

Modifying the medicament dosing protocol can comprise modifying a carbohydrate ratio.

In some embodiments, modifying the medicament dosing protocol comprises modifying a glucose target.

The controller can comprise a PID feedback-driven controller, a Kalman equation-driven controller, an extended Kalman filter controller, a regression tree-driven controller, a recurrent neural network-driven controller, a feed-forward neural network-driven controller, a support vector machine-driven controller, a self-organizing map-driven controller, a Gaussian process-driven controller, a genetic algorithm and program-driven controller, or a deep neural network-driven controller.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows an exemplary insulin delivery system.

FIG. 2 is a flow chart of an exemplary method of delivering insulin.

FIG. 3 is a graph showing uncompensated error contribution over time.

FIG. 4 is a graph showing compensated error contribution over time.

FIG. 5 is a prediction and update table of an EFK system.

FIG. 6 shows a prediction and update process.

FIG. 7 is a PID timeline of the prediction and update process.

FIG. 8 shows a PID prediction and update process.

FIG. 9 is a diagram showing an exemplary neural network implementation.

FIG. 10 is a diagram showing detail of predictive error of dosing adjustments of the neural network implementation of FIG. 9.

### DETAILED DESCRIPTION

Described herein is an insulin delivery system that is configured such that any user-initiated removal and subsequent insertion of an in-dwelling catheter (either infusion set, patch pump delivery cannula, or infusion port) can be accompanied by a reset, modification, or reinitialization of the AID algorithm. This reset, modification, or reinitialization can compensate for an otherwise potential over-delivery of insulin at new insertion sites. This reset, modification, or reinitialization can also use a calculated error to more accurately deliver insulin with continued use of the system.

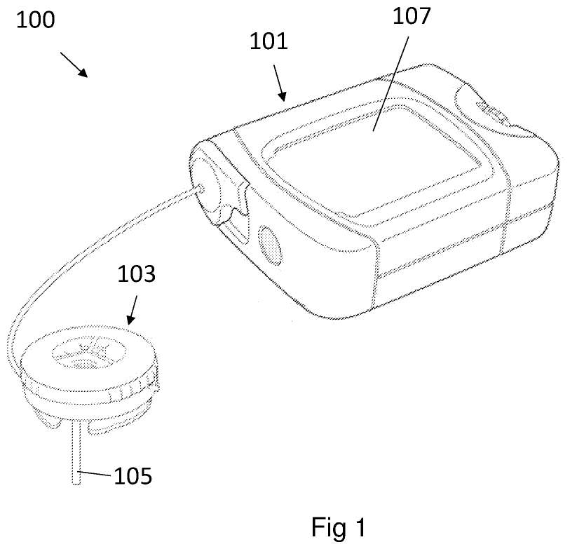

Referring to FIG. 1, in some embodiments, an insulin delivery system 100 can include a pump 101 and an infusion set base 103 configured connected to the pump 101 and to adhere to the patient's skin. The base 103 can include a cannula 105 configured to be inserted into the subcutaneous tissue for delivery of medicament (e.g., insulin) therethrough. The cannula 105 can be configured to remain in the subcutaneous tissue and delivery medicament therethrough for over 3 days, such as 5-10 days. Exemplary cannulas are described in International Application No. PCT/US2016/053118, filed on Sep. 22, 2016, titled “CONTINUOUS SUBCUTANEOUS INSULIN INFUSION CATHETER,” now PCT Publication No. WO 2017/053572, International Application No. PCT/US2018/025712, filed on Apr. 2, 2018, titled “HELICAL INSERTION INFUSION DEVICE,” now PCT Publication No. WO 2018/184012, and International Application No. PCT/US2019/060602, filed on Nov. 8, 2019, titled “LINEAR INSERTION DEVICE WITH ROTATIONAL DRIVE,” now PCT Publication No. WO 2020/097552, the entireties of which are incorporated by reference herein.

Further, the pump 101 can include a controller 107. The controller 107 can be configured to implement an automated insulin delivery (AID) protocol or algorithm to maintain the patient's blood glucose level at a target amount. The AID protocol used by the controller 107 can include a standard AID protocol updated to account for a change in cannula, as described herein. Exemplary standard AID protocols are described in U.S. Pat. No. 10,420,489B2, 10,173,007B2, EP2193814A1, US20170189614A1, CA2950966C, US20190388015A1, US20190053742A1, and EP2350895B1, the entireties of which are incorporated by reference herein.

Although the system 100 is shown in FIG. 1 as a tubed insulin pump system, it should be understood that the system can have an alternative design such that the indwelling catheter 105 and the controller 107 are part of a patch pump, a smart-pen, an implantable pump, or an external control device.

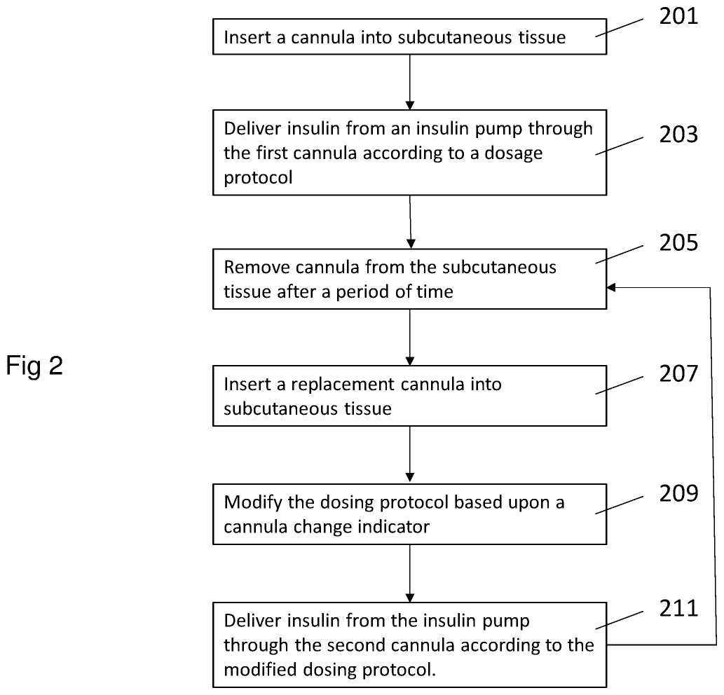

Referring to FIG. 2, use of the system 100 can include, at step 201, attaching the base 103 to the patient and inserting the cannula 105 into subcutaneous tissue. At step 203, insulin can be delivered from the pump 101 through the cannula 105 according to a dosage protocol implemented by the control 107. At step 205, after a period of time (e.g., greater than 3 days, such as 5-10 days), the cannula 105 can be removed from the subcutaneous tissue. At step 207, a replacement cannula 105 can be inserted into subcutaneous tissue. At step 209, the controller 107 can modify the dosage protocol based upon a cannula change indicator. At step 211, insulin can be delivered from the pump 101 through the cannula 105 according to a modified dosing protocol that accounts for the fact that the cannula 105 was changed (e.g., to account for increased insulin sensitivity at the new insertion site). The process can then be repeated (i.e., the cannula can be removed after a period of time, the protocol modified, etc.).

As noted with respect to FIG. 2, the controller 107 can be configured to modify the dosing protocol based upon an infusion cannula change indicator. For example, when the infusion set base 103 is removed from the patient and the cannula 105 switched for a new cannula (and then placed and activated again), the system 100 can automatically modify the dosing protocol. The automatic modification can be performed either by independent user action (e.g., by pushing a button on the pump 101 to indicate that a change was initiated) or as recommended or detected by the controller 107 itself. The infusion cannula change indicator can be, for example, detection of a pump mechanism rewind, detection of an insulin reservoir/cartridge change, detection of a tubing prime, detection of a cannula prime, installation of a new patch pump, user announcement of a set change via pump interface, or user confirmatory response to query about set change after any of the above.

As is further noted with respect to FIG. 2, when the in-dwelling catheter 105 is changed, the controller 107 (e.g., using the automated delivery algorithm) can calculate a dosing protocol. In some embodiments, the dosing protocol can be calculated based on data obtained prior to the change of catheter 105. Because the new insertion site, however, may not have the same insulin sensitivity as the previous insertion site (e.g., may have a higher insulin sensitivity), an automated correction factor can be implemented by the controller 107 based upon the change indicator. Thus, the controller 107 can implement an automated correction factor into the AID by introducing an additional term into the dosing equation and/or adjusting an existing term in the dosing equation. This additional term may be in the form of an independent matrix object in the Kalman equation and/or additional operators acting on a PID controller, fuzzy logic controller, a model predictive controller, a regression tree-driven controller, a recurrent neural network-driven controller, a feed-forward neural network-driven controller, a support vector machine-driven controller, a self-organizing map-driven controller, a Gaussian process-driven controller, a genetic algorithm and program-driven controller, or a deep neural network-driven controller.

In one static model-driven example, the controller 107 can include a sensor-driven (CGM) dosing algorithms that applies Kalman filters and/or extended Kalman filters (EKF) to estimate time-varying coefficients of the patient-specific state-space model. In the Kalman model, gain is the relative weight given to the measurements and current state estimate, and can be “tuned” to achieve a particular performance. With a high gain, the filter places more weight on the most recent measurements, and thus follow them more responsively. With a low gain, the filter follows the model predictions more closely. At the extremes, a high gain close to one will result in a more jumpy estimated trajectory, while a low gain close to zero will smooth out noise but decrease the responsiveness. It should be noted that the main difference between a Kalman filter and the extended Kalman filter is that the extended Kalman filter obtains predicted state estimate and predicted measurement by the nonlinear functions f(xk−1) fxk1 ð [symbol] ; uk1 and h xð [symbol] k, respectively. Further, in some embodiments, the controller 107 can use an extended Kalman filter with the addition of a matrix object that maintains an aggregate historical dataset of insulin consumption over time wherein the dataset is dynamically refreshed based on an event-driven user interaction, specifically the replacement of an in-dwelling catheter by the user (infusion set).

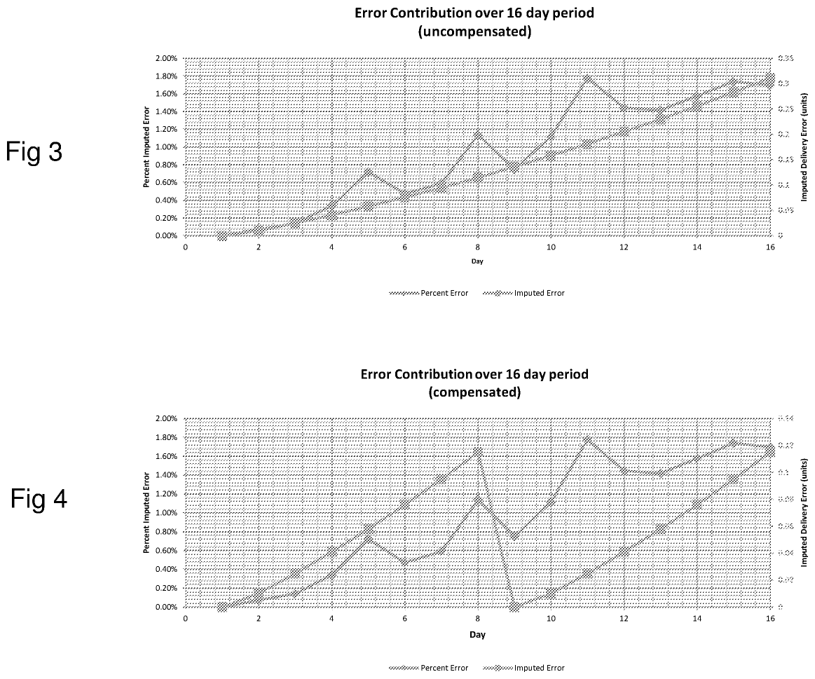

That is, inherent in Kalman filter function is the forward-looking error correction computed from historic data collection and processing. In the insulin delivery model, this manifests as an increasing deviation from “true” when CGM-derived data drive forward-dosing decisions. As the infusion site decreases sensitivity to insulin over time, the CGM measurement of blood glucose drives the Kalman equation to deliver ever-increasing insulin dosage to overcome the diminished tissue uptake capability. This is demonstrated by a simple model-derived graph shown in FIG. 3. The graph in FIG. 3 assumes that the infusion begins to lose sensitivity to insulin infusion on Day 3, which can introduce a compounding error into the Kalman calculation. The phenomenon of sensitivity loss can occur at any time after insertion. This automated insulin dosage calculation remains therapeutic over the duration-of-life of the in-dwelling catheter, thereby supporting appropriate insulin levels based on CGM measurement.

When a set change is initiated, either by independent user action or as recommended by error-correcting programming embodied in an insulin pump, the user can interact with the controller 107 to indicate (and/or the controller 107 can automatically determine) that a new in-dwelling catheter 105 has been inserted. This determination can activate a transition event to the introduction of the additional error-correcting term to reset and/or modify the dosing protocol, reflective of the set and/or cannula change, to slow the perceived error response. This is demonstrated by a simple model-derived graph shown in FIG. 4.

While insulin can be used to lower glucose, glucagon can be used to raise glucose levels. Accordingly, standard AID protocols can include, for example, both a dosing function (e.g., for insulin or glucagon) and a notification function (e.g., to notify a user to supplement with glucagon). The error-correction and/or modification to the protocol described herein (e.g., performed by the controller 107) can take a variety of forms related to dosing or notification. Modifications to the dosing algorithm can include changing an amount or timing of insulin delivery or changing alarms associated with hypoglycemia.

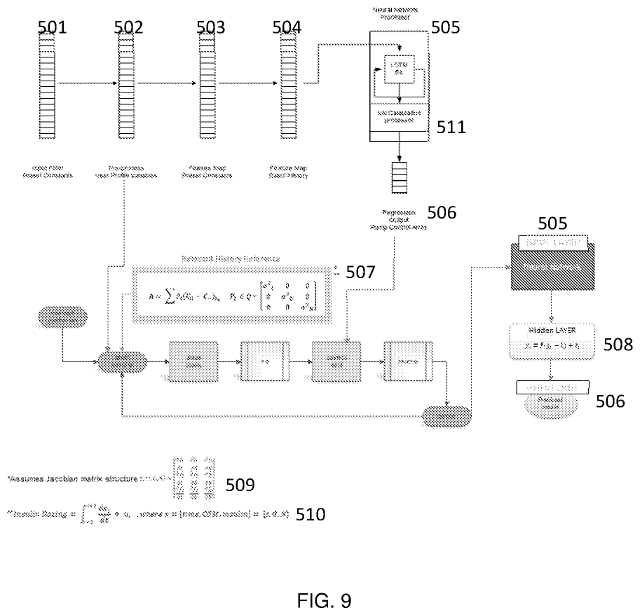

For example, the controller 107 can utilize the heuristically-derived glucose history array to dynamically define a low-glucose threshold. Increasing the threshold will drive the predictive control AI to a faster response protecting the user from hypoglycemia by forcing immediate pump control. This algorithm-derived control response will enable pump control to stop delivery, suspend delivery, start or stop the “suspend before low” and “alert before low” controls and/or user display/alert/alarm settings. This can be a control setting embedded in the algorithm feature map. (FIG. 9).

In some embodiments, the controller can temporarily increase the low limit that is associated with alerting users to potential low glucose (i.e., be more proactive in alerting the user of a pending low glucose). The controller can temporarily increase the low limit that is associated with suspending delivery if a low is predicted or occurring (i.e., stop the pump sooner). The controller can temporarily enable the “suspend before low” feature and/or the “suspend on low” feature if not enabled. In some embodiments, the controller can temporarily enable the “alert before low” feature and/or the “alert on low” on a pump if not enabled.

In some embodiments, the controller can increase the glucose level needed for delivery suspension from current level (e.g., increase from 70 mg/dL).

The controller can utilize the heuristically-derived dosing history array to dynamically define the pump control prohibition for control of the delivery suspension time period. Decreasing or eliminating control prohibition of delivery will support a more dynamic (faster) AI algorithm response to decreasing glucose levels. This can be a control setting embedded in the algorithm feature map. (FIG. 9).

In some embodiments, the controller can temporarily shorten the time limit that prohibits multiple suspend on low events from happening. The controller can temporarily eliminate any prohibitions of multiple suspend on low events from happening.

The controller can dynamically control the look-ahead predictive period to allow for controlled events. For example, if the normal user profile setting is typically 30 minutes ahead, it may look 45 or 60 minutes ahead for some time period following a site change. This can be a control setting embedded in the algorithm feature map. (FIG. 9).

The controller can dynamically control the default high and low glucose limit settings in the pump auto-mode control process. This can be a control setting embedded in the algorithm feature map (FIG. 9) that drives pump control.

In some embodiments, the controller can decrease the “safe basal” rate that the system has calculated based on prior history.

The controller can increase the reduction of basal delivery when low glucose event is predicted.

The controller can control default event time limit settings (duration and frequency) for glucose calibration requests by dynamically changing the threshold sensitivity control of minimum delivery rate and maximum delivery rate. This can be a control setting embedded in the algorithm feature map (FIG. 9) that drives user notification.

The controller can control default event time limit settings (duration and frequency) for glucose calibration requests by dynamically changing the threshold sensitivity control of CGM-derived measurement and finger-stick glucose measurement. Decreasing the threshold sensitivity control will trigger calibration requests with greater frequency (shorter time interval) supporting more frequent data acquisition driving increased response sensitivity. This can be a control setting embedded in the algorithm feature map (FIG. 9) that drives user notification.

In some embodiments, the controller can decrease the difference between entered blood glucose value and the measured CGM value that forces another blood glucose entry (e.g., a difference of less than 35%, such as 25%, can be utilized).

The controller can utilize user-input spot glucose value entry as a term to modulate the bolus calculation processor by dynamically changing the threshold sensitivity control of CGM-derived measurement and finger-stick glucose measurement. For example, increasing insulin bioavailability time (faster acting insulin) results in lower dosage recommended correction boluses. This can be an array generation/modification function embedded in the neural network calculation processor in the map. (FIG. 9).

In some embodiments, the controller can increase the insulin sensitivity value used by the bolus calculator when blood glucose values are entered as part of the bolus input, resulting in decreased correction bolus recommendation.

The controller can utilize the heuristically-derived dosing history array to dynamically define the insulin response array. Calculated and stored PKPD data will support a more precise AI algorithm response to glucose levels. For example, increasing insulin bioavailability time (faster acting insulin) results in lower dosage recommended correction boluses. This can be an array generation/modification function embedded in the neural network calculation processor in the map. (FIG. 9).

The controller can dynamically control default event function to override user notification settings in the event of a predicted or real-time control limit threshold violation. For example: override user-suppressed alert or alarm setting in the event of a hypo or hyperglycemic event.

The controller can utilize the heuristically-derived glucose history array to dynamically define a high-glucose threshold. Increasing the threshold will drive the predictive control AI to a slower response protecting the user from hypoglycemia by suppressing immediate pump control. This can be a control setting embedded in the algorithm feature map. (FIG. 9).

The controller can utilize the heuristically-derived glucose history array to dynamically control threshold limits associated with automated basal and bolus delivery. Modification of threshold values will drive the predictive control AI to a faster response by forcing immediate pump control. This algorithm-derived control response will enable pump control to stop delivery, suspend delivery, start or stop the “suspend before low” and “alert before low” controls and/or user display/alert/alarm settings. This can be a control setting embedded in the algorithm feature map. (FIG. 9).

In some embodiments, the controller can raise the limit associated with decreases in basal delivery (e.g., raise the limit above 112.5 mg/dL).

The controller can raise the limit associated with suspension of insulin delivery (e.g., raise the limit above 70 mg/dL).

The controller can reduce the maximum allowable basal rate limit (e.g., less than 3 u/h if no CGM within 20 minutes to a lower value).

The controller can increase the correction factor or insulin sensitivity used in any dosing calculation.

The controller can increase insulin duration (e.g., to greater than 5 hours), resulting in a higher calculation of IOB and thus lower correction boluses.

The controller can increase the lower target blood glucose (e.g., to greater than 110 mg/dL).

The controller can decrease the personal profile basal rate delivery (i.e., what basal is delivered when CGM is in target range).

The controller can dynamically control default limit settings in the pump auto-mode control process. These limits are stored as named variables in the User Control array(s). Examples: any one or any combination of maximum insulin delivery rate, upper limit glucose target, personal profile correction factors, total daily insulin and user weight. This is a control setting embedded in the algorithm feature map (FIG. 9) that drives pump control.

In some embodiments, the controller can increase the upper limit glucose target (e.g., to greater than 160 mg/dL).

The controller can decrease the % of total correction bolus calculated based on the personal profile correction factor and predicted CGM reading (e.g., to less than 60%).

In some embodiments, the controller can increase the personal profile correction factor used by the automatic correction bolus calculation.

In some embodiments, the controller can decrease the maximum automatic correction bolus (e.g., to less than 6 units).

The controller can dynamically control the target glucose limit settings in the pump auto-mode control process when the body physical state is altered (e.g., in periods of exercise, in periods of sleep, etc.). This algorithm-derived control response will enable pump control to modify insulin delivery based on target glucose value during suspended delivery states. This is a control setting embedded in the algorithm feature map (FIG. 9) that drives pump control.

In some embodiments, the controller can increase target range used during sleep (e.g., to greater than 112.5-120 mg/dL) and/or the target range during exercise (e.g., to greater than 140 to 160 mg/dL).

The controller can increase the glucose value associated with delivery suspension during exercise (e.g., to greater than 80 mg/dL).

In some embodiments, the controller can change the user weight utilized in dosing calculations.

The controller can decrease the total daily insulin value utilized in dosing calculations and dosing limits.

In some embodiments, the controller can increase the gain on a standard protocol that would release glucagon in response to dropping glucose values and/or other indications of pending hypoglycemia. Exemplary standard protocols for the release of glucagon are described in US 20180220942A1, the entirety of which is incorporated by reference herein.

In some embodiments, the controller 107 can be configured to modify a Kalman or extended Kalman filter calculation by the addition of a new Jacobian matrix that is introduced into the calculation when a transition trigger condition or state is achieved. This can be illustrated by the following equation (Eq. 1) modeling new simplified interpretation of the EKF, stating that a change in the function of next-state prediction over time is calculated based on the deviation in the over-all history compared to the next point in time, while also considering the effect of error calculation, uₜ, on the equation.

$$Insulin Dosing = {\int }_{t - 1}^{t + 1} \frac{{dx}_{t}}{dt} + {u}_{t} , \\ where x = [ time , CGM , insulin ] = [ t , G , N ] \\ {x}_{t} = f ( {x}_{t - 1} , {u}_{t - 1} ) + {𝓌}_{t - 1} = > prediction function \qquad ( Eq . 1 )$$

$${z}_{t} = 𝒽 ( {x}_{t} ) + {𝓋}_{t} = > measurements i . e . CGM readings at time t$$

Where xₜ is the current state calculated by the function ƒ of the previous state, xₜ₋₁, and the control input, uₜ₋₁. [symbol] is the measurement function correlating the current state, xₜ, to the measurement zₜ; [symbol] ₜ₋₁ and [symbol] ₜ are Gaussian noises for the process model and the measurement model with covariance Q and R, respectively; [symbol] ₜ₋₁˜ [symbol] (0, Q) where Q is a Gaussian matrix with the standard deviations σₜ, σ_{G}, σ_{N} of the process model; and [symbol] ₜ˜ [symbol] (0, R) where R is a Gaussian matrix with the standard deviations σₜ, σ_{G}, σ_{N} of the measurement model.

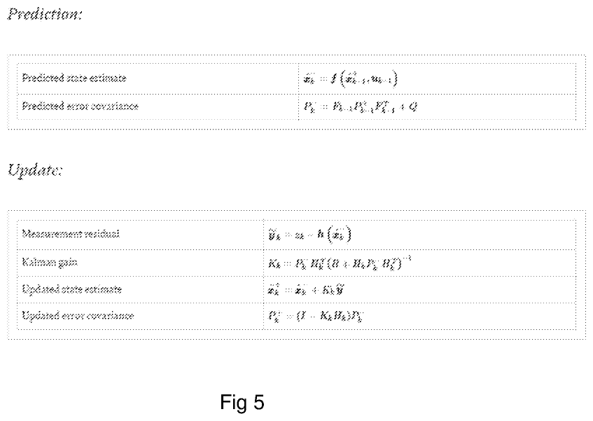

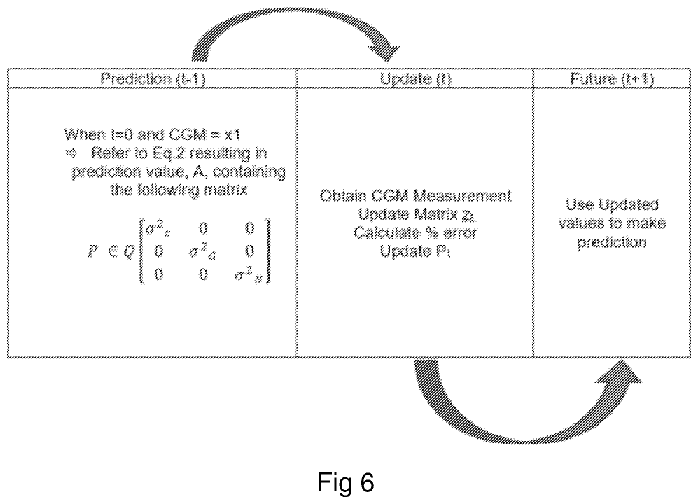

The controller 107 in this embodiment implies the theory of “transition trigger event” occurring when an indwelling infusion set has been worn for x amount of time represented by t. As previously explained, this event will be triggered when a user makes an indication of a new set being inserted, or the pump system determined the need for a new set. This event trigger indicates a cannula or infusion set change is needed, leading to the gate for the new system calculation this invention is disclosing. In order for the gate to be activated, the user may confirm a change of the infusion set, triggering the start of wear duration (t=0) and a CGM measurement needs to be obtained, represented as an x1 value. A combination of the previous conditions will lead to the new prediction system to run, as shown in FIGS. 5 and 6. For the transition trigger event, t=days for duration of wear is complete (e.g., t=7). For the gate, t=0 indicated by user's confirmation on pump AND CGM=x₁, and the insulin dosage prediction is calculated by xₜ=x₂.

In other embodiments, the controller 107 can implement a PID-based calculation modified by the addition of a new matrix introduced into the calculation when a transition trigger condition or state is achieved. On initialization, the PID sequence can use either historical data points to support the prediction or can take the most recent data point as an input.

Many current pump systems are not intended for extended use, and so do not contemplate compensating for increased insulin dosage requirements associated with extended use. In current dosing algorithms (e.g., those used in current pump systems), the starting point of any process gets setup using the initialization step, which, traditionally, either does not take into account any historical data points to support the prediction, or takes the most recent data point as an input, which may not be the most accurate.

In contrast, in some embodiments, the innovation described herein narrows down the purpose of each data and takes into consideration recent historical data points only relevant to the event occurring at the starting point. For example, the algorithm takes into account data from a time period (e.g., 1 day, 2 days, 12 hours, 6 hours, etc.) surrounding previous event triggers.

As noted, in this embodiment, the controller 107 can narrow down the purpose of each dataset and utilizes recent historical data points only relevant to the event trigger occurring at the starting point t=0. One of the major contributors to the newly developed matrix to collect all relevant historical data of the infusion set usage is the use of Jacobian matrix. For the system described herein, the use of logic of the Jacobian matrix in combination with the Gaussian matrix knowledge can allows for the collection of the deviation of function output over time in terms of multiple variable, in this case: time (t), Glucose measurement (G), and insulin infusion dose (N), as seen in the following matrix representation:

$${𝕁}_{f} ( t , G , N ) = [ \begin{matrix}\frac{\partial {f}_{1}}{\partial t} & \frac{\partial {f}_{1}}{\partial G} & \frac{\partial {f}_{1}}{\partial N} \\ \frac{\partial {f}_{2}}{\partial t} & \frac{\partial {f}_{2}}{\partial G} & \frac{\partial {f}_{2}}{\partial N} \\ \frac{\partial {f}_{3}}{\partial t} & \frac{\partial {f}_{3}}{\partial G} & \frac{\partial {f}_{3}}{\partial N}\end{matrix} ]$$

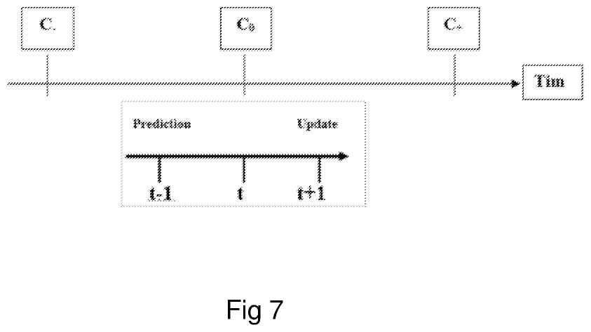

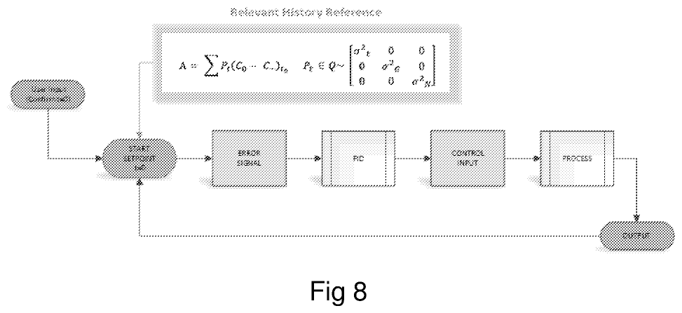

In the diagrammatic representations of FIGS. 7 and 8, the equation models the incorporation of relevant averages of historical data and the associated percentage error matrix in a Gaussian logic in terms of time, glucose measurement, and insulin infusion dose. To best the new calculations resulting in an updated prediction matrix, the below equation was created (Eq. 2) utilizing the previously discussed EKF algorithm. In some embodiments, equation 2 can be incorporated into the PID system currently used by the controller 107 (and the AID algorithm) by implementing the utilization of summing the relevant average data collected previously, given the nomenclature “Relevant History Reference.” Noting that relevant average refers to the average of data collected on a previous period of time relevant to previous insertion days and calculated error percentages on those days (i.e. all available previous t=0 s). A combination of Eq. 2 and a PID system maximizes the system's efficiency and finesse in predicting user's required dose, minimizing user's life risk. In this equation, Eq. 2, the terms used are defined as below:
C₋=A duration of time previous to the current.
C₀=current duration of time.
C₊=future duration in time.
t−1=the prediction state, occurs in the time frame prior to the occurrence of the event.
t=current time point when the event is occurring, the algorithm updates based on real-time measurements taken at this point in time, resulting in error calculations and finer predictions.
t+1=upcoming time point, in which updated predictions will be utilized.
P̅ₜ=Average of percentage error of previous error estimation, as calculated by the Extended Kalman Filter (EKF) method; can be a matrix entity.
Q=covariance Gaussian matrix with the standard deviations σₜ, σ_{G}, σ_{N} of the process model

$$\begin{matrix}A = \sum {\overline{{P}_{t}} ( {C}_{0} - {C}_{-} )}_{{t}_{0}} & {P}_{t} ∈ Q \sim [ \begin{matrix}{σ}_{t}^{2} & 0 & 0 \\ 0 & {σ}_{G}^{2} & 0 \\ 0 & 0 & {σ}_{N}^{2}\end{matrix}\end{matrix} ] \\ Event - driven PID Equation \qquad Eq . 2$$

Advantageously, the systems described herein can automatically adjust for the increased perfusion/absorption that may occur after a new cannula has been inserted into the subcutaneous tissue (i.e., relative to the previous cannula just prior to removal). The systems described herein can thus help prevent hypoglycemic excursions for the user.

### Neural Network Implementation

Embodiments of neural network implementations are described below.

The predictors (or inputs) form the bottom layer, and the forecasts (or outputs) form the top layer.

The coefficients attached to these predictions are obtained by a linear combination of the inputs. The weights are used in the “learning algorithm” to minimize a “cost function” such as the MSE.

Weights need to be restricted to prevent too many constraints; parameter restricting the weights are often set to be equal to 0.1.

The weights are updated using observed data collection, resulting in a variable of randomness in the predictions produced by a neural network. Therefore, the neural network processor will be optimized with every activation using variable starting points values, and the results are averaged. Historical results as well as clinical data aid in the first couple activations to drive the protocol closer to optimization and predictions accuracy.

### Neural Network Autoregression

In a neural network autoregression or NNAR model, lagged values from the time dependent series and estimated values are used as inputs to the neural network. In such model NNAR(x,y) format is used to indicate there are (x) inputs and (y) nodes in the hidden layer.

For forecasting one prediction output ahead, the available historical data is used. For forecasting two predictions ahead, we use the one-step forecast as an input, along with the historical data. The general equation for forecasting is as below

yₜ=ƒ(yₜ−1)+εₜ

where yₜ−1 is a vector containing values from the time dependent series, and ƒ is a neural network with (y) hidden nodes in the middle layer. The error series {εt} 508 (FIG. 9) is assumed to be homoscedastic (and possibly also normally distributed). In some embodiments, like in this protocol, the error calculations from 507 will be used as value for this error constant, which in this case can be portrayed as an array of historical error values.

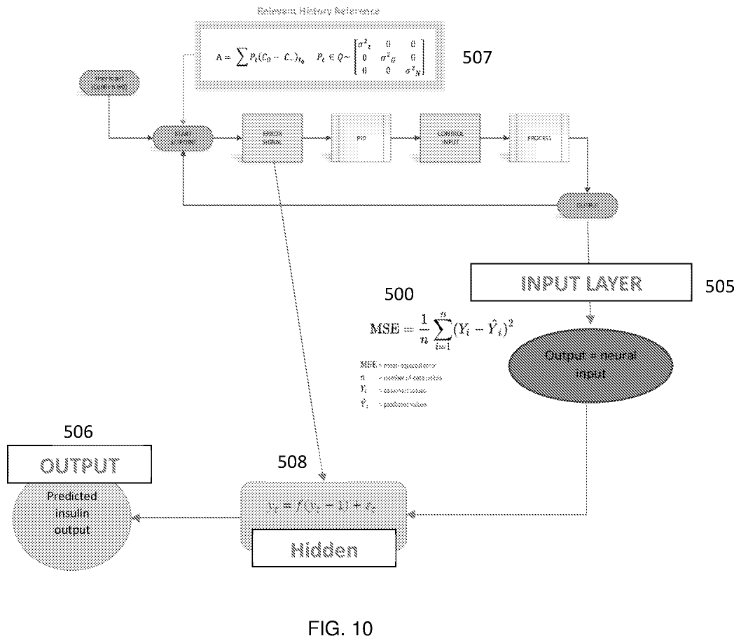

Neural network calculations can be implemented in this system by taking output of the PD-centered system and using it as an input to the “input layer” of the neural network model. The neural network modeling technique consists of three main layers: input layer, hidden layer, and output layer. The input layer consists of the time data points being inputted into the system per a linear regression equation; for implementation into the previously discussed model, the input layer will have the outputted insulin dosing predictions used as inputs into this layer. In this layer, also exists coefficients known as “weights” based on the error calculated (e.g., in the Mean Squared Error algorithm 500 (FIG. 10)) to promote learning from previously calculated error percentage from historical data learning The second layer in a neural network is known as “hidden layer” where non-linear regression model is accounted for based on the variables presented in the inputted forecast (e.g, 505 (FIG. 9)).

In this layer further error percentage is calculated (e.g., using MSE process 500 (FIG. 10)).

By implementing neural network within the previously discussed model, the response prediction of insulin gets closer to accuracy and accounts for the error calculated previously.

Referring now to FIG. 9, a schematic diagram of the neural network implementation is shown. At “input filter” 501, pump signals, including but not limited to, indicating a set change, prime cannula, and/or a remind activation, along with a user input confirming removal of the first cannula from the subcutaneous tissue after a period of time and inserting a second cannula into subcutaneous tissue.

At 502, the protocol uses user profile variables and past performance history prior to proceeding with protocol. The contributors collected to proceed include but are not limited to delivery method limits, high medication dose threshold, low medication dose threshold. Additionally, any user-set limits and preference will be implemented in function 502 contributing to minimizing over-delivery or under-delivery. With continuous use of the protocol, additional parameters are added, and new limitations are learned from the data.

In feature map 503, historical data of measured glucose value and amount of relatively infused insulin is stored in a 3-D matrix of time, CGM measurement input, and insulin at that point in time and space. With each system activation, further data in the same matrix layout is stacked to the array creating additional layers of identified history point in terms of time (t), glucose (G), and insulin (N) at point of activation (to). In the mentioned matrix above, with each layer of collected data array at activation point, also exists an additional layer of error, relative to the associated matrix, calculated after each activation.

The matrix is a component of the sum of error calculations from all the previous events where the “process” was activated.

After collection of time, glucose, and insulin data at point of activation, another set of the same data is collected post-activation and inputted into an MSE equation to calculate error percentage of the dosing adjustments. The outputted error value along with the relative data array is appended into the matrix maintained in FIG. 9, shown at 507.

At 505, the neural network processor (NNP) is activated where the predicted output from series 501, 502, 503, 504 is used as input into the NNP consisting of a Long Short Term Memory (LSTM) processor with at least 3 layers of data analysis, data predictors, and error correction equations. Continuous use of 505 builds a larger database of the activation protocol's error calculations; with every protocol activation, the calculated error percentage in the LSTM processor decreases and aims to reach an asymptotic value nearing zero. Error signal calculated previously in 503 and 504 is used as a “weight” in the Hidden Layer contributing to the accuracy of the prediction (FIG. 10). Min function of 505 is to use the protocol output with the estimated error calculation prior to deployment, obtain the real measured value after activation and deployment, calculate the mean square error between the observed and predicted value, then update the database with calculated error value to be inputted in the upcoming activation calculations creating the control arrays 506.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-72. (canceled)

73. A subcutaneous medicament delivery system comprising:
a pump;
a cannula attached to the pump; and
a controller, the controller configured to:
automatically calculate a medicament dosage protocol based upon a blood glucose level;
modify the medicament dosage protocol when an event driven signal indicative of an infusion cannula change is detected; and
use neural network calculations utilizing calculated error data to modify the medicament dosage protocol.

74-77. (canceled)

78. The system of claim 73, wherein the step of automatically calculating is performed with a Kalman filter.

79. The system of claim 73, wherein the step of automatically calculating is performed with a model predictive control (MPC).

80. The system of claim 73, wherein the controller is a PID feedback controller.

81. The system of claim 73, wherein the controller is a fuzzy logic controller.

82. The system of claim 73, wherein modifying the medicament dosing protocol comprises modifying a historical setting according to previous cannula changes.

83-92. (canceled)

93. The system of claim 73, wherein the controller comprises a PID feedback-driven controller, a Kalman equation-driven controller, an extended Kalman filter controller, a regression tree-driven controller, a recurrent neural network-driven controller, a feed-forward neural network-driven controller, a support vector machine-driven controller, a self-organizing map-driven controller, a Gaussian process-driven controller, a genetic algorithm and program-driven controller, or a deep neural network-driven controller.

94. The system of claim 73, wherein the controller is configured to retain historical performance data and use retained historical performance data to modify the medicament dosage protocol.

95. The system of claim 94, wherein the controller is configured to retain historical performance data relevant to one or more previous cannula changes, and only use the relevant historical performance data to modify the medicament dosage protocol.

96. The system of claim 95, wherein the relevant historical performance data comprises data from a set time period surrounding one or more previous cannula changes.

97. A subcutaneous medicament delivery system comprising:
a pump;
a cannula attached to the pump; and
a controller, the controller configured to:
automatically calculate a medicament dosage protocol based upon a blood glucose level;
modify the medicament dosage protocol when an event driven signal indicative of an infusion cannula change is detected; and
retain historical performance data and use retained historical performance data to modify the medicament dosage protocol.

98. The system of claim 97, wherein the controller is configured to use relevant historical performance data relevant to one or more previous infusion cannula changes.

99. The system of claim 98, wherein relevant historical performance data comprises data from a time period surrounding one or more previous infusion cannula changes.

100-103. (canceled)

104. The system of claim 97, wherein the step of automatically calculating is performed with a Kalman filter.

105. The system of claim 97, wherein the step of automatically calculating is performed with a model predictive control (MPC).

106. The system of claim 97, wherein the controller is a PID feedback controller.

107. The system of claim 97, wherein the controller is a fuzzy logic controller.

108. The system of claim 97, wherein modifying the medicament dosing protocol comprises modifying a historical setting according to previous cannula changes.

109-118. (canceled)

119. The system of claim 97, wherein the controller comprises a PID feedback-driven controller, a Kalman equation-driven controller, an extended Kalman filter controller, a regression tree-driven controller, a recurrent neural network-driven controller, a feed-forward neural network-driven controller, a support vector machine-driven controller, a self-organizing map-driven controller, a Gaussian process-driven controller, a genetic algorithm and program-driven controller, or a deep neural network-driven controller.
